(19) United States
(12) Patent Application Publication
(10) Pub. No.: US 2022/0313143 A1
(43) Pub. Date: Oct. 6, 2022

(54) VIRTUAL IMMERSIVE SENSORIMOTOR DEVICE AND METHODS TO DETECT NEUROLOGICAL IMPAIRMENTS

(71) Applicant: University of Mississippi Medical Center, Jackson, MS (US)

(72) Inventors: Jennifer Reneker, Clinton, MS (US); William Pruett, Madison, MS (US)

(21) Appl. No.: 17/710,984

(22) Filed: Mar. 31, 2022

Related U.S. Application Data
(60) Provisional application No. 63/169,186, filed on Mar. 31, 2021.

(51) Int. Cl. A61B 5/00; G06T 15/00; H04N 13/344; G06F 3/01; A61B 5/16; A61B 5/11

(52) U.S. Cl. CPC A61B 5/4064; G06T 15/00; H04N 13/344; G06F 3/012; G06F 3/013; A61B 5/163; A61B 5/7267; A61B 5/1114; G06T 2210/41

(57) ABSTRACT

Provided herein are a method of detecting neurological impairment and a device for testing sensorimotor control to detect neurological impairment. The method includes positioning a head-mounted display on the user's head, the head-mounted display placing the user in a virtual or augmented reality environment; presenting the subject with a software-generated object in the virtual or augmented reality environment; providing instructions directing the user to execute one or more sensorimotor activities relating to the software-generated object within the virtual or augmented reality environment; recording data during execution of the one or more objectives; and determining if there is a neurological impairment based upon the recorded data. The device includes a head-mounted display, one or more processors, and one or more hardware storage devices having stored thereon computer-executable instructions which are executable by the one or more processors to display a virtual immersive environment in the head-mounted display.

### RELATED APPLICATIONS

This application claims priority from U.S. Provisional Application Ser. No. 63/169,186, filed Mar. 31, 2021, the entire disclosure of which is incorporated herein by this reference.

### TECHNICAL FIELD

The presently-disclosed subject matter generally relates to articles and methods for detecting neurological impairment. More specifically, the presently-disclosed subject matter relates to a virtual immersive sensorimotor device and methods to detect neurological impairments.

### BACKGROUND

Sensorimotor control is vital to the acquisition and execution of skilled human movement. Impairments of sensorimotor control can be present anywhere within this complex and integrative system, which involves many neurological processes. On the afferent side of an efficiently functioning sensorimotor control system, the vestibular system detects angular and linear acceleration of the head, providing feedback about movement and eliciting ocular and postural reflexes; the visual system (including perception and oculomotor control) is used to plan whole body movements in a feed-forward manner; and the somatic system provides information across all joints and muscles in the body to permit proprioceptive and kinesthetic awareness. These inputs of sensory information are integrated and interpreted in the central nervous system, combined with sub-conscious and executive cognitive processes, and result in directed efferent output to enable intended actions through precise motor control. The maintenance of functional postures and skilled movements are only possible through efficient sensorimotor control. However, neurological or sensorimotor control impairments are detectable in a variety of neurological diseases, conditions, and traumatic events.

Neurological events causing either transient, chronic, or progressive neurological impairments can be divided into two broad categories; acquired brain injuries and neurological diseases. Acquired injuries and neurological diseases damage the brain's neuronal activity and affect the physical integrity, metabolic activity, or efficiency of the nerve cells. An acquired brain injury (ABI) is an injury that is not hereditary, congenital, or degenerative but rather is the result of a traumatic or non-traumatic event.

A traumatic brain injury (TBI) is an event, triggered by an external force, which alters how information is processed by the brain. A TBI may or may not cause structural damage, altering the physical anatomy of the brain. Resultant neurological impairments after a TBI may be transient, in the case of most concussions, or chronic, in the case of most moderate to severe TBIs. TBIs are often associated with sports activities, motor vehicle accidents, falls, military operations, and assaults.

Non-traumatic acquired brain injuries also result in neurological impairments and structural changes. Lack of oxygen, inadequate perfusion, exposure to toxins, and infectious diseases often cause non-traumatic brain injuries. In some cases, observed neurological impairments are not the result of an injury and are temporary in nature, including those induced by the incident use of drugs, alcohol, low blood sugar, or sleep deprivation. The other major category of neurological impairments are those derived from neurological diseases or degenerative conditions which include, but are not limited to, Alzheimer's disease, Parkinson's Disease, dementias, and epilepsy.

The detection of neurological injury is performed by a physician or other healthcare provider skilled in neurologic examination techniques. To provide a complete evaluation of a structural injury to the brain, a physician will use diagnostic imaging tools such as cranial computed tomography (CT), magnetic resonance imaging (MRI), and positron emission tomography (PET) scans. These tests can be invasive and cannot precisely define the extent and types of neurological impairments experienced by a person with the brain injury. Rather, these tests are used to identify the areas of injury, extent and type of tissue damage, and resolution over time. These diagnostic tests are limited in that they cannot be used immediately after a potential injury occurs. Second, the correlation between the structural injury identified and the extent and types of neurological impairment experienced by the individual is not linear.

Accordingly, there remains a need for improved devices and methods to detect neurological impairment.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments, the presently-disclosed subject matter is directed to a method to detect neurological impairment of a user using examination of sensorimotor control, the method including positioning a head-mounted display on the user's head, the head-mounted display placing the user in a virtual or augmented reality environment; presenting the subject with a software-generated object in the virtual or augmented reality environment; providing instructions directing the user to execute one or more sensorimotor activities relating to the software-generated object within the virtual or augmented reality environment; recording data during execution of the one or more objectives; and determining if there is a neurological impairment based upon the recorded data, where the method is at least partially implemented by a computer system including the head-mounted display; one or more sensors for recording the data; one or more processors; and one or more hardware storage devices. In some embodiments, the sensorimotor activities include one or more of the following smooth pursuit or convergence eye movement; saccadic eye movement; peripheral visual acuity; object discrimination; gaze stability; head-eye coordination; and cervical neuromotor control. In some embodiments, the instructions are provided to the user as audio or visual instructions. In some embodiments, the data includes one or more of object data, response data, or symptom data. In some embodiments, the placing of the user in a virtual or augmented reality environment includes displaying a three-dimensional environment to the user through the head-mounted display. In some embodiments, the head-mounted display includes a screen for each eye.

In some embodiments, the step of determining if there is a neurological impairment based upon the recorded data includes generating a sensorimotor index from the recorded data; assigning a test grade based upon the sensorimotor index; and determining if the user has a neurological impairment based upon the test grade. In some embodiments, the sensorimotor index is generated using statistical computation or machine learning artificial intelligence computation. In some embodiments, the step of determining if there is a neurological impairment based upon the recorded data includes generating a user sensorimotor index from the recorded data; comparing the user sensorimotor index to at least one other sensorimotor index; and determining if the user has a neurological impairment based upon the comparison with the at least one other sensorimotor index. In some embodiments, the at least one other sensorimotor index is selected from the group consisting of a previous sensorimotor index generated from the user's prior data, a designated population without a known neurological impairment, and a designated population with a known neurological impairment. In some embodiments, the user sensorimotor index and at least one other sensorimotor index are generated using statistical computation or machine learning artificial intelligence computation. In some embodiments, the machine learning artificial intelligence makes a determination about the type of impairment for each sensorimotor activity or a combination of one or more of the sensorimotor activities when completed together;

In some embodiments, the one or more sensors comprise a hand tracking sensor for tracking position and movement of the user's hand within the virtual immersive environment relative to other objects displayed within the virtual immersive environment. In some embodiments, the one or more sensors comprise an accelerometer head sensor, a gyroscope head sensor, an eye tracking device, a magnetometer, or combination thereof. In some embodiments, the user is neurologically impaired due to trauma, vascular, aging, or other physiological processes.

Also provided herein, in some embodiments, is a device configured for testing sensorimotor control to detect neurological impairment of a user, the device including a head-mounted display including a screen for each eye, the screens being configured to display a virtual immersive environment to the user; an audio system configured to provide instructions to the user for executing one or more sensorimotor activities within the virtual immersive environment; an eye tracking system; and at least one sensor configured to record data during the sensorimotor activities; one or more processors; and one or more hardware storage devices having stored thereon computer-executable instructions which are executable by the one or more processors to cause the display of the virtual immersive environment and recording of the data. In some embodiments, the at least one sensor comprise an accelerometer head sensor, a gyroscope head sensor, an eye tracking device, a magnetometer, or combination thereof. In some embodiments, the one or more sensorimotor activities comprise one or more of smooth pursuit, saccade, near point convergence, peripheral vision acuity, object discrimination, gaze stability, head-eye coordination, or cervical neuromotor control. In some embodiments, the eye tracking system is selected from the group consisting of a single tracker for both eyes and two trackers for each eye.

Further provided herein, in some embodiments, is a hardware storage device having stored thereon computer-executable instructions which are executable by one or more processors of a computer system to cause the computer system to record a sensorimotor performance metric of a user; display a virtual immersive environment to the user by way of the head-mounted display with one screen for an eye; provide instructions directing the user to execute one or more sensorimotor activities within the virtual immersive environment, and recording object, response, or symptom data of the user during execution of the one or more sensorimotor activities; based on the recorded object, response, or symptom data, determine sensorimotor performance metric of the user indicating the user's proficiency in executing the one or more sensorimotor activities; use statistical testing to determine if there is an impairment of sensorimotor control.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 shows a schematic illustrating a computing environment in which a virtual immersive environment is generated for providing sensorimotor control testing to an individual.

FIG. 2 shows a schematic illustrating a method of determining an individual's sensorimotor control using the computer systems of FIG. 1.

FIG. 3 shows a screen shot illustrating a smooth pursuit test in the virtual immersive environment showing the pattern taken by the object as it moves across the screen.

FIGS. 4A-B show screen shots illustrating a binocular near-point convergence test in the virtual immersive environment. (A) An object where it begins optically distant from the individual. (B) The object where it ends optically close to the individual.

FIG. 5 shows a screen shot illustrating a peripheral vision test as displayed on a device for the examiner. The central and side objects are presented on the display and in the virtual immersive environment.

FIG. 6 shows a screen shot illustrating a cervical neuromotor control test as displayed on a virtual immersive environment. The bulls-eye target with the pointer (black dot) is controlled by the position of the individual's head while wearing the head-mounted device.

FIG. 7 shows a screen shot illustrating the symptom assessment as displayed on a device for the examiner. The symptom assessment is displayed in the center.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong. Where reference is made to a URL or other such identifier or address, it is understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise. When open-ended terms such as “including” or ‘including, but not limited to” are used, there may be other non-enumerated members of a list that would be suitable for the making, using or sale of any embodiment thereof.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

“AI sensorimotor control index” means a numeric representation or score on a sensorimotor control test, calculated from object data and the individual's response data collected discretely at a specific time or over a time period, using a machine learning or an artificial intelligence (AI) function trained to distinguish impaired from non-impaired individuals on a sensorimotor control test.

“Functional position” describes a posture or movement that permits multiple planes of motion, requiring the individual to control multiple joints to maintain stability to perform a functional activity. This includes, but is not limited to, sitting, standing, walking, jumping, running, throwing, kicking, bending, turning, or other movement activity.

“Global index” means a numeric representation or score calculated from two or more sensorimotor control indices.

“Global grade” means a qualitative determination of the overall degree and type of neurological impairment demonstrated by an individual on two or more sensorimotor control tests. This can be based on the value of the numeric representation from the global index.

“Neurological event” means those incidents or actions that cause an alteration in the brain's neuronal activity, which affects the physical integrity, metabolic activity, or efficiency of the nerve cells. A neurological event can be associated with a specific disease pathology, injury or trauma, or exposure to a chemical, toxin, or any other situation that influences brain processes. Furthermore, the event can be a discrete incident, transient or chronic, or can be progressive in nature.

“Neurological or sensorimotor control impairment” means a limitation in the capacity of the nervous system to function efficiently. Impairment may be present in one or more of the following areas or combination thereof: memory, cognition, sensory processing, motor skills, organizational abilities, or information processing.

“Object” means a distinct visual presentation of a computer generated representation of a thing or things, likeness of a person, or other representation in the virtual immersive environment, to which a response can be elicited or attention can be given and a specified action can be directed for a sensorimotor control test. Objects have a position and may have additional characteristics including but not limited to, color, size, shape, and highlighting.

“Objective” means a specified action with a purpose or goal for a sensorimotor control test as directed by an audio, visual, or both instruction.

“Object data” means a digital representation in the virtual immersive 3D environment of a sensorimotor control test, in which the positions and characteristics of objects are recorded at fixed time intervals. This data describes the location, movement, position, or visual characteristics of one or more objects displayed to the individual during the sensorimotor control test.

“Response” means the set of all actions of the individual reacting to, moving, positioning, or acting on one or more objects during the testing of sensorimotor control in a virtual immersive environment. This includes, but is not limited to, the actions of an individual to choose or designate the position or presence of an object, such as a trigger activation or individual selection, head position, gaze direction, head orientation, visible area of the pupil, postural sway, or hand position. These actions may be subconscious, reflexive, or generated through cognitive processes.

“Response data” means the digital representation of the response of the individual to a sensorimotor control test in the virtual immerse 3D environment at a specific time or over a period of time. This data is captured at the same fixed intervals as the object data. The data includes, but is not limited to, the position of the testing device in 3D space, the position and gaze direction of the individual's eyes, and the selection of certain test objects by gaze fixation or the activation of a trigger.

“Sensorimotor control” means the global ability of an individual to receive sensory information and use the sensory information along with cognitive processes to complete skilled motor actions, which can be measured. Upon receiving the sensory information, the central nervous system of the individual processes and integrates these inputs, which may occur fully or partially subconsciously, to direct movement of the eyes, head, body, hands, and feet.

“Sensorimotor control test” means an examination of an element of sensorimotor control. Sensorimotor control tests include an objective for the individual to perform. Sensorimotor tests include, but are not limited to, smooth pursuit, pro and anti-saccades, near-point convergence, peripheral vision, object discrimination, gaze stability, head-eye coordination, cervical joint proprioception, and kinesthetic awareness.

“Statistical sensorimotor control index” means a numeric representation or score on a sensorimotor control test, calculated from object data and the individual's response data, collected discretely at a specific time or over a time-period, using a statistical test, to distinguish impaired from non-impaired individuals on a sensorimotor control test.

“Sensory information” means afferent input, used by an individual, from one or more of visual, vestibular, or joint/muscle mechanoreceptor signals elicited during completion of a sensorimotor control test.

“Test grade” means a qualitative value representing the degree of sensorimotor control of an individual on a sensorimotor control test. Each “test grade” is defined by a range of possible index scores. This can be based on the value of the numeric representation from the statistical or AI sensorimotor index.

“Symptom data” means any or all data describing the individual's self-assessment of symptoms during the completion of a sensorimotor control test in a virtual immersive environment. The individual's ability to record his/her perception of symptoms with a numeric, visual analog scale, or pictorial representation describing severity is required to contribute to this type of data.

“Symptom index” means a numeric representation or score on one or more sensorimotor control tests, calculated from the individual's symptom data, collected discretely at a specific time, using an artificial intelligence function trained on a designated population to quantify the likelihood that an individual is included in a designated population.

“Symptom grade” means a qualitative value representing the severity of symptom report from individual on one or more sensorimotor control tests. Each “symptom grade” is defined by a range of possible symptom index scores.

“Virtual immersive environment” means a virtual, three-dimensional environment displayed to the individual by way of a head mounted device and/or other display device. Although a virtual immersive environment may be illustrated herein using two dimensional figures and related description, it is understood that the virtual immersive environment will provide the individual an actual three-dimensional environment to the individual by way of one screen to an eye.

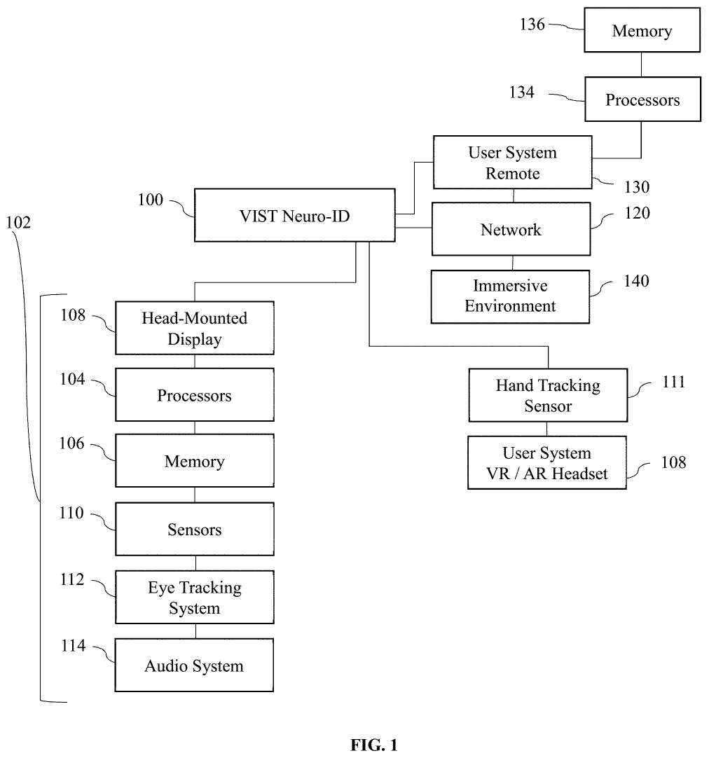

Provided herein, in some embodiments, are articles for detecting neurological impairment in a subject. For example, in some embodiments, as illustrated in FIG. 1, the article includes a computing device 100 configured to provide a virtual immersive environment to test the sensorimotor control of an individual (e.g., deliver sensorimotor control tests) to detect neurological impairment. The computing device 100 includes a user system 102 and optionally a user remote system 130 and/or an immersive environment server 140. This computing device 100 may also be referred to herein as the Virtual Immersive Sensorimotor Testing Device (VIST).

The user system 102 includes one or more processors 104 and memory 106 (e.g., in the form of one or more hardware storage devices). Suitable processors 104 include, but are not limited to, microprocessors, video processors, application specific integrated circuits (ASICs), and systems on a chip (SOACs). Suitable types of memory 106 include RAM, ROM, DRAM, SRAM, and MRAM, which may be stored on a hardware storage device such as disk media, electronic, or other like bulk, long-term storage or high-capacity storage medium. Types of firmware include static data or fixed instructions, BIOS, system functions configuration data or other routines used for operation. In some embodiments, the user system 102 also includes a head-mounted display (HMD) 108, which is also referred to interchangeably herein as a head-mounted extended reality device, configured to display a virtual immersive environment to the user when worn by the user. The term HMD should be understood to be synonymous with similar terms referring to similar display devices such as “headset,” “VR device,” “VR display,” “AR device,” “AR display,” and the like. In some embodiments, the HMD covers substantially all of the user's visual field and may permit simultaneous visual input and interaction with the physical environment and the virtual environment

In some embodiments, the user system 102 also includes one or more sensors 110. Suitable sensors include, but are not limited to, sensors for measuring the position and/or movement of the head, hand, and/or other body parts. Particular examples of sensors 110 include, but are not limited to, accelerometers (e.g., to detect postural sway or reaction movement of the head and/or hand to stimuli), gyroscopes (e.g., to measure the position of the head and/or hand), and magnetometers (e.g., to measure physical orientation of the user). Sensors 110 associated with the hand 111 may also be utilized to allow the user to select objectives, navigate in-display menus, make in-display selections, and interact with one or more virtual objects displayed within the virtual immersive environment. Collision detection (i.e., detecting the intersection of one or more objects or object paths) between the user's hand and one or more virtual objects may also be determined by way of sensor(s) 110 of the hand. Although described herein primarily with respect to measuring head and hand movement, the disclosure is not so limited and expressly includes measuring movement of other body parts, as well as sensorimotor activities that relate to other body parts.

Additionally or alternatively, the user system 102 may include an eye tracking system 112 and/or an audio system 114. In some embodiments, the eye tracking system 112 measures eye position and/or movement of one or both eyes in response to sensory stimuli, including visual and/or vestibular stimuli. For example, eye tracking may be utilized to measure elements including, but not limited to, the location of the user's gaze, the speed, accuracy, latency, or smoothness of the user's eye movements, or combination thereof. Eye tracking may also be used to count the number of blinks and measure the pupil size during the activity. In some embodiments, the eye tracking system 112 includes a digital camera that enables the capture of eye, and specifically pupil, movement in response to stimuli. The digital camera preferably operates at a frame rate of at least 120 Hz and may capture the eye focus of the user at approximately the same rate for recording by the user system 102 and/or the remote user system 130. An example of an eye tracking device that may be utilized in the disclosed user system 102 is the commercially available system by HTC with the trade name Vive Pro Eye that contains a Tobii eye tracker. In common use, such systems track eye movement to a region being viewed for the purpose of limiting the rendered content of the 3D information displayed to the user in order to permit faster response time to the viewer of the 3D environment. In contrast, the user system 102 utilizes such eye tracking systems to measure the aforementioned parameters of eye movement, including in some cases the ability to find a specific target, and the reaction time to find object. In some embodiments, the audio system 114 includes one or more speakers for communicating audio to the user and/or one or more microphones for receiving audio from the user.

In some embodiments, the computing environment includes a user remote system 130. The user remote system 130 is a computer device separate from the user system 102, and includes its own processor(s) 134 and memory 136. In some embodiments, the user remote system 130 processes the inputs and outputs of the virtual immersive environment displayed by the user system 102. For example, the user remote system 130 may have greater computer processing power than the onboard processor(s) 104 of the user system 102, and the processing required to run the virtual immersive environment and/or analyze user responses may be divided among the user system 102 and user remote system 130 according to the needs of the particular hardware at hand. The user remote device 130 may be a mobile computer device, such as a mobile telephone, tablet, or the like, a desktop personal computer, or other suitable computer device.

In some embodiments, the computing environment 100 includes an immersive environment server 140. The immersive environment server 140 may also be utilized to contribute to the computer processing required to run the virtual immersive environment. Additionally or alternatively, the user remote system 130 and/or the immersive environment server 140 connect to the user system 102, each other, and/or other remote computer devices, such as those associated with healthcare providers, trainers, coaches, and the like by network 120. In some embodiments, the network 120 includes one or more of a cellular network, Local Area Network (“LAN”), a Wide Area Network (“WAN”), or the Internet, for example. The network 120 may thus include wireless and/or wired connections between the various components of the computing environment 100. Physical connection interfaces include USB, HDMI, DVI, VGA, fiber optics, DisplayPort, Lightning connectors, Ethernet, and the like. The connection of the user remote system 130, the immersive environment server 140, the user system 102, and/or other remote computer devices allows these remote devices to send instructions to the user remote system 130 and/or user system 102 to control the virtual immersive environment and to receive results according to the user's interaction with the virtual immersive environment. For example, a healthcare provider may be able to remotely select or adjust a training/therapy regimen (e.g., adjust type of activities and/or difficulty) within the virtual immersive environment, monitor the user's execution of the associated sensorimotor activities in real time, adjust instructions on the fly, and provide remote, real time assessments and/or feedback.

Operating the user system 102 (e.g., by running appropriate software) includes presenting a subject with a virtual immersive environment where sensory stimuli are provided in a controlled manner and one or more sensorimotor activities may be performed by the subject. In some embodiments, the user system 102 provides analysis of the subject's motor and/or visual responses to the sensory stimuli by way of the motion sensors 110 and/or eye tracking system 112. For example, as the user moves in response to the sensorimotor activities performed within the virtual immersive environment, the associated movement data is collected and analyzed for parameters such as accuracy, stability, precision, timing, coordination, and the like.

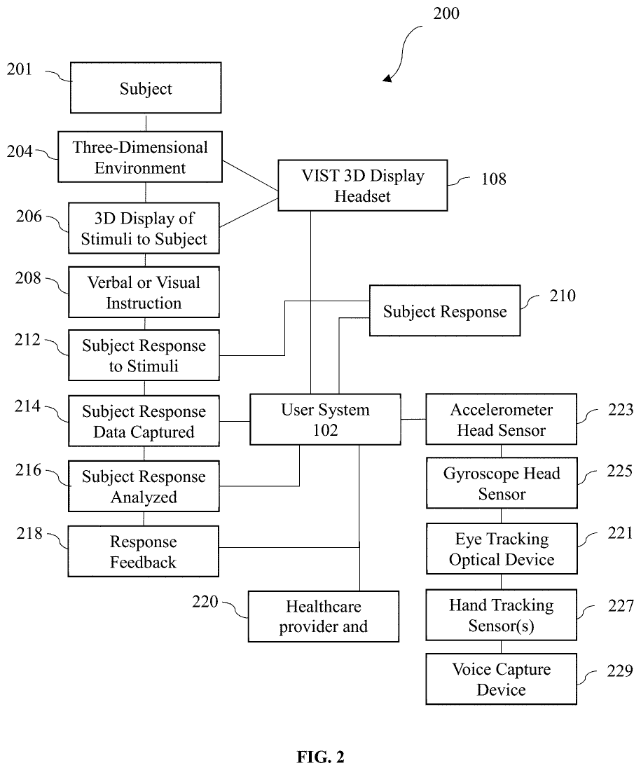

Also provided herein, in some embodiments, are methods for detecting neurological impairment in a subject. In some embodiments, the method includes using a sensorimotor control test for the detection of a neurological impairment in a subject. In some embodiments, using a sensorimotor control test for the detection of a neurological impairment includes first placing a subject in a virtual or augmented reality environment; presenting the subject with a software-generated object in the virtual or augmented reality environment; providing the subject with one or more objectives to perform on, or in relation to, the object in the sensorimotor test; and collecting response data while the subject completes each objective. In the first step, the subject may be placed in the virtual or augmented reality environment by any suitable method or device. For example, in one embodiment, as illustrated in the method 200 shown in FIG. 2, the subject 201 is placed in the virtual or augmented reality environment using the computing device 100 (FIG. 1) according to one or more of the embodiments disclosed herein. In another embodiment, placing the subject in the virtual or augmented reality environment includes positioning the HMD 108 on the subject's head. In some embodiments, the HMD contains a visual display for each eye (i.e., a separate display lens for each eye), an eye tracker device to measure the movement of the eyes, a series of sensors to measure up to 6 degrees-of-freedom movement, and an audio device for each ear. The display may generate virtual, augmented, or mixed reality. In some embodiments, the eye tracker includes a single eye tracker for measuring the movement of both eyes. Alternatively, in some embodiments, the eye tracker includes one or more eye trackers for each eye. In one embodiment, for example, the HMD is a virtual reality (VR) headset, with one display screen per eye, producing a three-dimensional (3D) environment 204. Optionally, one or more hand controllers with sensors measuring hand movements can be included with the HMD 108.

In the next step, after the subject is placed in the virtual immersive 3D environment, the computing device 100 (e.g., the HMD 108) displays one or more objects 206 to the subject in the immersive 3D environment. The objects being displayed and/or the behavior of the objects is determined based upon the specific sensorimotor control test being performed. In some embodiments, the one or more objects may be displayed in an expected, or unexpected manner; continuously moving, discretely moving, or discontinuously non-moving manner depending upon the objective of the sensorimotor control test. For example, in one embodiment, the one or more objects change their position in an anticipatory or non-anticipatory fashion in the immersive 3D environment relative to the depth perception or spatial orientation of the individual. Additionally or alternatively, in some embodiments, one or more attributes of the object presented to the subject may be changed. These attributes include, but are not limited to, number, size, color, placement, movement, speed, or other attributes of the object, and may be changed in an anticipatory or non-anticipatory manner. In some embodiments, the changing of the attributes is controlled by software, an external examiner, or both.

In the third step, the subject is provided visual and/or audio instruction 208 informing the subject how to complete the one or more objectives. Each objective requires the subject to perform a visual and/or physical activity on, or in relation to, the one or more objects displayed in the immersive 3D environment in order to engage their sensorimotor control system. In some embodiments, the objective of the test may include one or more of a motor (i.e., movement) response of the eyes, head, body, or hands. For example, in one embodiment, the individual uses one or more hand controllers with tracking sensors to follow the motion of the object, find the object, or select an object as instructed. Other instructions may include the individual placing their body in a functional position with their feet and hands in specific positions including balancing on unstable surfaces or moving in the real environment.

When receiving the instructions and/or completing the objective(s), the subject may be in a functional position, including sitting, standing, walking, running, jumping, throwing, kicking, bending, turning, or other movement activity. In one embodiment, the subject is preferably sitting or standing. Optionally, the individual may be in a challenging standing posture, including tandem, narrow, or single leg stance while completing the test. Although described herein primarily as being provided to the subject after the object is presented, the disclosure is not so limited and the instructions relating to the one or more objectives may be provided at any time before the subject acts (e.g., before being placed in the virtual immersive 3D environment, before the object is presented, etc.).

Together, the object(s) and objective(s) form one or more sensorimotor tests. Suitable sensorimotor control tests include, but are not limited to, smooth pursuit, pro and anti-saccades, near-point convergence, peripheral vision, object discrimination, figure-ground perception, gaze stability, head-eye coordination, cervical joint proprioception, kinesthetic awareness, and combinations thereof. These sensorimotor control tests may be utilized in the method to identify deficits associated with various aspects of sensorimotor control, including oculomotor control, visual perception, vestibular reflexes, cervical proprioception, balance, reaction time, and symptomatic response to each test.

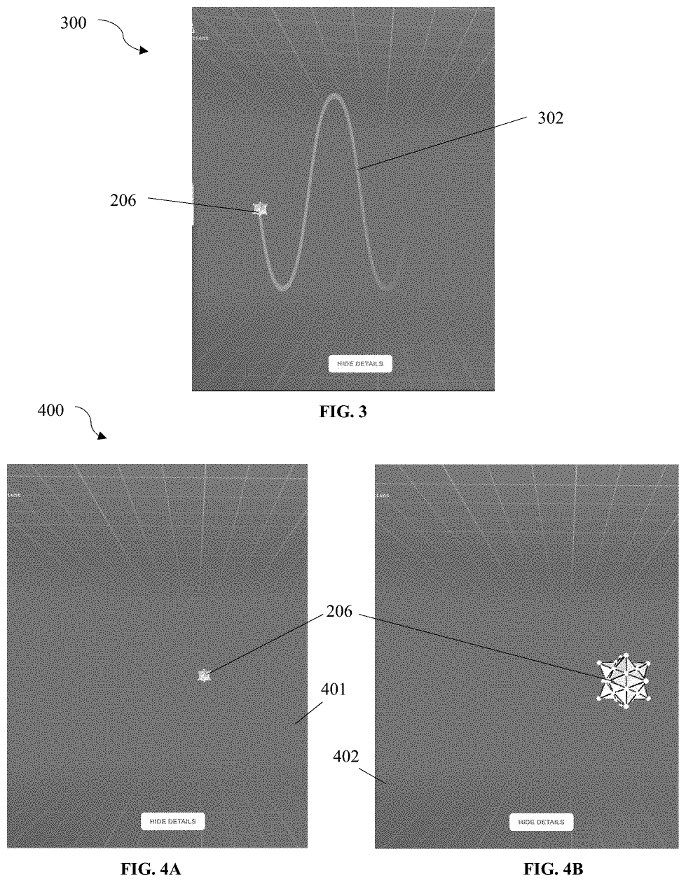

In some embodiments, the smooth pursuits sensorimotor control test is used for identifying impairments associated with binocular smooth pursuit oculomotor control. In some embodiments, the smooth pursuits sensorimotor control test includes generating the virtual immersive environment 300 illustrated in FIG. 3. In such embodiments, the individual is presented with a 3D moving object 206 in the VIST device. The objective is that the individual follows the path of the moving object with his/her eyes while keeping the head still and in midline. This object begins on the left and move across the screen in a sinusoidal or other random 3D pattern 302 and is be repeated with the object moving from right to left. This may be repeated at unpredictable velocities. For further testing of smooth pursuits, the individual can be instructed to fixate on a central object and a moving object may come in, unpredictably, from the right or left side and move directly to the opposite direction. The moving object hits the center at 100 ms after it is presented, cross over midline and continue to the position off midline and stop, stay visible as a stationary object in that off-midline position, and finally disappear. Trials move left to right and right to left with the trials on each side having the movement of the object increasing in speed after each one.

In some embodiments, the pro- and anti-saccades sensorimotor control test is used to identify impairments associated with pro- and anti-saccadic oculomotor control. In such embodiments, the individual is presented with a 3D object in the virtual immersive environment, which appears in the center of the visual field while wearing the VIST device. Then, rapidly and unexpectedly, a second object appears off midline on the left or right; or above or below center. Based on the color of the central object, the objective is to quickly move the eyes towards (pro-saccade) or away from (anti-saccade) the peripheral object, while keeping the head still. Presentation of the object is only in one location at a time (center, right, left, up or down). The HMD unpredictably varies whether a pro- or anti-saccade is requested and where the object is located.

In some embodiments, the binocular near-point convergence sensorimotor control test is used to identify deficits of near-point binocular ocular convergence. In such embodiments, as illustrated in FIG. 4, a virtual immersive environment 400 is configured to present the individual with a 3D moving object in the device. The objective is for the subject to keep their eyes fixed on an object that begins in the center, appearing optically distant 401, and moves towards the individual's eyes 402. The speed of the object should be constant but can vary randomly from test to test, and the individual's head should remain in midline. The individual is asked to click on the trigger of the manual controller if they perceive two objects instead of one as it moves closer to the eyes. Regardless, the object should continue moving to the closest point near the face as possible. This should be repeated more than one time.

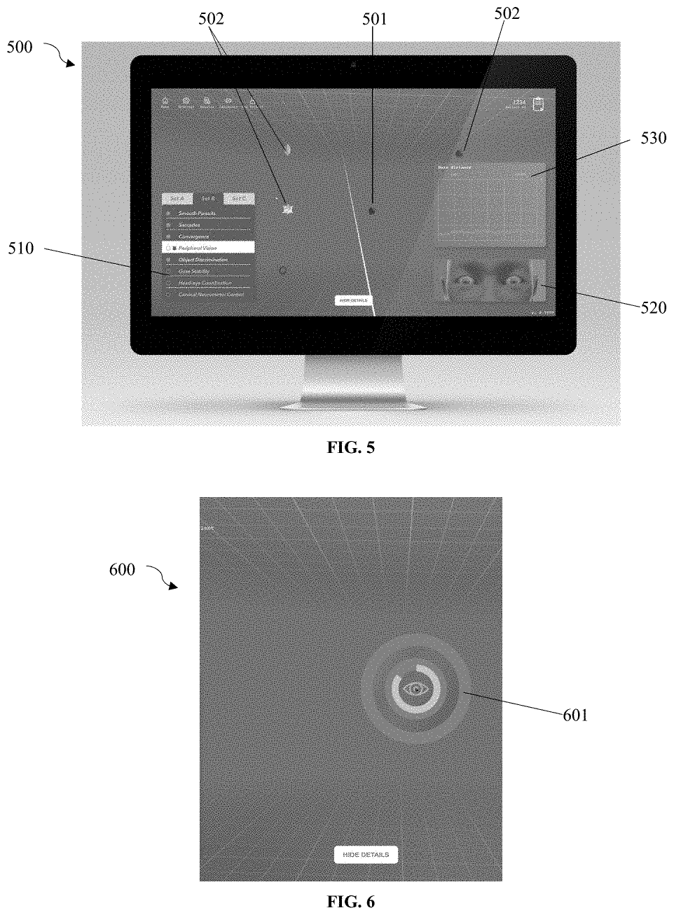

The peripheral vision sensorimotor control test is used to identify impairments associated with peripheral visual acuity, perception, and ability to shift central and peripheral attention without moving the eyes. Referring to FIG. 5, in some embodiments, a virtual immersive environment 500 is configured to present the individual with a display of a 3D central object 501 and a series of objects on the left and right sides 502 while wearing the device. Additionally, the computer dashboard 510 for the examiner includes a presentation of all the sensorimotor control tests and an avatar 520 and graph 530 showing the individual's eyes while taking the tests. The color and/or shape of the central object changes randomly each second as the colored and/or shaped objects on the sides move laterally. The objective is to maintain visual gaze on the central object, while the individual uses their peripheral vision to identify the corresponding colored/shaped object's position on one of the sides and with a manual selection by the hand controller. The number of correct choices within the timeframe is recorded.

The object discrimination sensorimotor control test is used to identify impairment in the ability to distinguish differences in two objects. The individual is presented with two objects and the objective is to move the eyes in the direction of the larger object. This is done multiple times with the location of the longer line varying unpredictably between left and right side and the lines becoming more alike in size.

The gaze stability sensorimotor control test is used to identify impairment associated with self-driven visual gaze stability. In such embodiments, the individual is presented with a 3D object, with the object location anchored to the device, regardless of head position. The individual is asked to turn their head left and right (headshake “no”) to the beat of an audible metronome, then move their head up and down (headshake “yes”) to the beat of an audible metronome. In some embodiments, the subjected is instructed to complete ten cycles of each motion, although any other suitable number of cycles may be used. The beats per minute, as set by the audible metronome, include any suitable number of beats per minute and can vary with each test. In some embodiments, the beats per minute is set at 180 beats per minute, so that with each beat the head should be in the opposite direction. The objective is to keep the eyes focused on the central object during head movement while keeping the desired speed of movement.

The head-eye coordination sensorimotor control test is used to identify deficits with eye movement coordination during simultaneous head movement. In such embodiments, one small 3D object is presented in a location that requires the individual to move their eyes and head to that location. After the subject moves to each location, an arrow directs them to the location of the next object and then disappears. The objective is to quickly and accurately move the head and eyes to align with the presented object. The movements include discrete horizontal, vertical, and diagonal movements in an unpredictable pattern.

The cervical neuromotor control sensorimotor control test is used to determine if there are impairments of cervical proprioceptive awareness when visual input is not available. Referring to FIG. 6, in some embodiments, a virtual immersive environment 600 is configured to display a bulls-eye target 601 to the individual on the headset screen with the head in midline. A pointer (indicating position of the head) relative to the center of the target indicates when the target is directly in the center. Then the screen goes black and the individual is asked to actively rotate their head all the way to the right and return to this midline position. When the individual believes that they are at midline, they press a manual button on a hand control and the pointer lights up indicating their ending position compared to the ideal (i.e., central starting) location. The process may be repeated any suitable number of time. For example, in one embodiment, the process is repeated 3 times to the right, followed by 3 times to the left, and then 3 times into cervical extension.

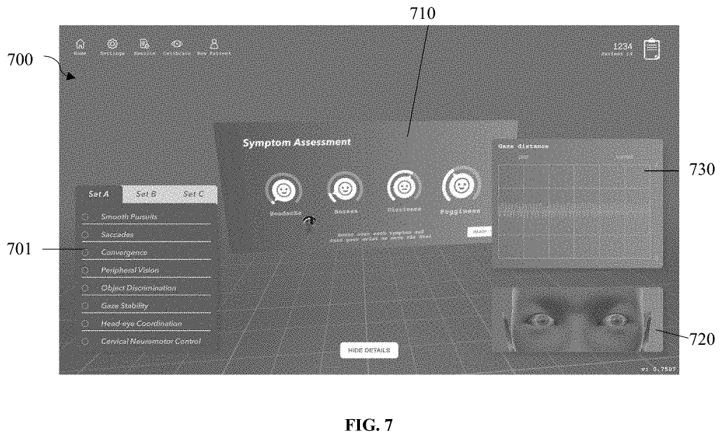

Turning to FIG. 7, in some embodiments, the virtual immersive environment 700 is projected from to a computer, which can be operated by a medical provider or examiner. In one embodiment, the dashboard is configured to show the names of the sensorimotor tests and which tests have been taken 701. This screenshot in FIG. 7 shows the symptom display in the VR headset 710, which changes on the computer in response to the selections made by the individual taking the test. The display also shows the individual's eye movements 720 on an avatar, in real time as a test is completed, along with a graphic of the eye movement 730.

Although the sensorimotor control tests are described herein primarily with respect to specific examples, the disclosure is not so limited and specifically includes various modifications thereto. For example, in some embodiments, the sensorimotor control tests may be modified by requiring completion in a different functional position (e.g., while walking to increase to increase the level of complexity and requirement of overall sensorimotor control).

In the fourth step, as the individual executes the objective(s) of the one or more sensorimotor control tests in the virtual immersive 3D environment 210, response data 212 are collected 214 by one or more sensors or devices. In some embodiments, the one or more sensors or devices are contained in the HMD and/or hand controllers. Suitable sensors or devices include, but are not limited to, an eye-tracker 221, head tracker, accelerometer 223, gyroscope 225, magnetometer, hand tracker 227, voice capture device 229, or combination thereof. For example, in one embodiment, continuously collected discrete data is generated during the completion of the one or more sensorimotor activities from one or more of an eye-tracker, accelerometer, gyroscope, magnetometer, or combination thereof. In another embodiment, symptom data is collected from the individual through self-report. In a further embodiment, the further object data, response data, or symptom data are generated from the software display of the object, head or body movement, hand movement/controller activation, eye movement/or characteristics of the eye, or a combination thereof.

In some embodiments, the response date includes one or more of the subject's motor responses to a sensorimotor control test. For example, in one embodiment, as illustrated in FIG. 2, a series of sensors and devices capture the motor response of individual's movements, speed and position, of their head, and eyes as the individual responds to the test in the immersive 3D environment. In some embodiments, the movement of the individual's head involves a 3D orientation of the head position represented by axes of pitch, roll, and yaw. The position of the head provides information on the motor response of the head to the objective of the sensorimotor control test. In some embodiments, the motor response of the individual's eyes, specifically pupil movement, to visual stimuli, is captured using an eye-tracker device. The eye-tracker provides information on where the eye is relative to the object or objects, characteristics of the eyes, and the timing of when the eyes respond to the object or objects in the 3D HMD display. In some embodiments, the eye-tracker captures eye-related data at 90 times per second or more during a typical response and the eye-related response data derived from the measurements from the eye-tracker are recorded by the headset user system or remote. Other suitable capture rates include, but are not limited to, between 45 Hz and 500 Hz, between 45 Hz and 200 Hz, between 90 Hz and 500 Hz, or any suitable combination, sub-combination, range, or sub-range thereof. Other sensors and devices capture the motor response of one or both hand movements, while another device captures the voice and verbal responses through a voice recognition software.

The response data may be stored on a computer storage device, which may be either on the HMD or any other suitable location or remote computer device (e.g., a separate computer in the same room, a separate computer at a remote location, or cloud based storage). In some embodiments, following collection, the response data is compared 216 to object data obtained from the sensorimotor control test to detect whether the subject has an impairment. In one embodiment, the response data and object data are compared by a computer software program. In another embodiment, the computer software program is executed by a computer processor on the HMD, at a remote computer device, or both.

In some embodiments, the response data and the object data are compared using statistical computation or by machine learning/artificial intelligence (AI) (e.g., random forest, gradient boosted trees, random vectors). In the first step for statistical or AI computation, the data is cleaned to eliminate data produced as an artifact from the measurement methods and data collected before a test begins or after it ends. In the next step, in addition to features extracted during the data collection and data cleaning, machine learning features are extracted from the cleaned object data and the response data collected discretely at a specific time or over a time-period. The features may include, but are not limited to, eye location, gaze fixation, blink length or frequency, gaze fixation defect, discrete Fourier transform, or nodes from the penultimate layer of a neural network. Additionally or alternatively, in some embodiments, the machine learning AI computation is performed by calculating a score from extracted features utilizing an AI trained to determine inclusion in a designated population. The designated population may be normal, generally impaired, or impaired in a specific way. In an alternate embodiment, a statistical computation is performed by comparing the values of extracted features in the sensorimotor control test for the individual and the expected features seen in a designated population. The software program generates a sensorimotor control index from the AI or statistical computation, which is used to determine the individual's inclusion in the designated population. Additionally or alternatively, in some embodiments, the AI may be used to extrapolate other diagnoses and/or different diagnoses.

In some embodiments, the tests provide an overall examination of how sensory information is being taken in, processed, and utilized by an individual to direct movement. Additionally or alternatively, in some embodiments, the tests may be utilized to measure changes in symptomatic presentation and identify how using these sensory systems impact neurologic stimulation. Furthermore, in some embodiments, the tests assist in the identification of specific neurological impairments or events to be monitored and/or treated. Still further, in some embodiments, the tests are used to monitor the recovery after a neurological event, including concussion or other injury associated with neurological impairment.

In some embodiments, the method also includes identifying a degree of sensorimotor control of a subject. For example, in one embodiment, the degree of sensorimotor control is identified using a sensorimotor control index. The sensorimotor control index is a numeric representation or score on a sensorimotor control test, which is calculated from object data and the subject's response data, collected discretely at a specific time or over a time-period. In some embodiments, the sensorimotor control index is generated based on a statistical computation or machine learning artificial intelligence (AI) software computation comparing the object data and response data of the individual while performing one or more of the sensorimotor control tests. Additionally or alternatively, in some embodiments, an individual is assigned a test grade based on the AI or statistical sensorimotor control index to describe the degree of sensorimotor control according to a specific sensorimotor control test. In some embodiments, the comparison is made relative to the values obtained for the preceding motor responses to determine improvement of motor responses to selected motor response evaluations of activities. In some embodiments, limitations or deficiencies may be present after a neurological event or may be described relative to the optimal level of neurological efficiency.

Further provided herein are methods of determining a type of neurological impairment. In some embodiments, determining a type of neurological impairment includes computation of two or more sensorimotor control indices using AI machine learning trained on a designated population without a known neurological impairment and a population of impaired individuals having a specific type of neurological impairment. Alternatively, in some embodiments, determining a type of neurological impairment includes statistical computation of two or more sensorimotor control indices for comparing a designated population without a known neurological impairment to a population of individuals with known type of impairment. The designated population in the AI machine learning and/or statistical computation embodiments may be normal, generally impaired, or impaired in a specific way.

In some embodiments, if two or more sensorimotor control tests are completed, a global index is calculated across two or more sensorimotor control indices. A global index may be generated each time the individual completes two or more sensorimotor control tests. The global index is stored on the HMD, remote computer device, or both, and may be used to compare differences in an individual neurological function at two or more times, either sequentially or at later times. The sensorimotor control index and global index is presented on a computer screen for the individual, healthcare providers, or others present with the individual or in a remote location to indicate the individual's test grade and neurological event probability. The indices are reported either visually or audio message as either actual data or an indication of impairment relative to a normal motor response. Alternatively, the reported motor response is compared relative to the individual's prior evaluation.

In some embodiments, the global index is used to determine a global grade. The global grade is used to identify the degree of neurological impairment. The global grade may be used on sequential tests on an individual over time to detect change, either reduced or improved, in a subject's degree of neurological impairment. For example, in one embodiment, identifying the degree of neurological impairment includes a comparison or multiple comparisons of a subject's global grade before and after a period of training. In another embodiment, identifying the degree of neurological impairment includes a comparison or multiple comparisons of a subject's global grade before and after rehabilitation for an impairment. In a further embodiment, the subject's global grade is compared to a collection of different types of neurological impairment indices from two or more subjects having a similar neurological impairment after training or rehabilitation.

In some embodiments, the subject is asked to rate their symptoms on a scale according to perceived severity before and after completion of a sensorimotor control test. This symptom data may be included in the response data or evaluated independently. In some embodiments, the symptom data of the subject prior to a sensorimotor control test is compared to the symptom data after completion of the sensorimotor control test to generate a symptom index. The comparison of symptom data to generate the symptom index is performed via a statistical computation or a machine learning/artificial intelligence (AI) computation. In some embodiments, this comparison is calculated by a computer software program, which is executed by a computer processor on the HMD and/or at a remote computer device. Other parameters that can be measured include, but are not limited to, physiological changes such as heart rate, blood pressure, cognitive indicators, oxygen content, or any other physiological parameter.

In some embodiment, the method includes evaluating the global and symptom indices to determine if an individual has sustained a neurological event. Traumatic events caused by external physical forces on a subject's brain, such as sport injuries, motor vehicle accidents, falls, assaults, or similar acts, may result in structural alterations. Additionally, non-traumatic events, including those which are either chronic or transient in duration, may result in structural alterations. For example, chronic non-traumatic events include, but are not limited to, a lack of oxygen, stroke, exposure to toxins, and infectious diseases affecting the brain structure. Alternatively, non-traumatic events causing transient neurological impairments include, but are not limited to, those induced by the incident of drug and/or or alcohol use, low blood sugar, or sleep deprivation. In some embodiments, the neurological impairments include those derived from neurological diseases or degenerative conditions which include, but are not limited to, Alzheimer's disease, Parkinson's disease, dementias, and epilepsy. In the latter neurological impairment, the global and symptom indices are able to predict the neurological diseases or degenerative conditions before traditional tests. A machine learning AI computation is performed by calculating a score from the global and symptom indices utilizing an AI trained to determine inclusion in a designated population. The designated population may be diagnosed with a specific neurological event. The global+symptom index is used to determine the individual's inclusion in the designated population.

Further provided herein are methods of detecting a specific area or areas of the central nervous system that have been affected by a neurological event. In some embodiments, the method includes using one or more sensorimotor control indices based on statistical computations or AI computations from one or more sensorimotor control tests to identify a specific area of damage in the central nervous system. The method for detection of a specific area of damage in the central nervous system may be determined by AI machine learning computation of one or more sensorimotor control indices using AI trained on a designated population and on individuals with a specific area of damage in the central nervous system.

Further provided herein are methods of treating or rehabilitating an individual having a neurological impairment. In some embodiments, the method includes treating or rehabilitating a neurological impairment detected by sensorimotor control tests using one or more sensorimotor control indices. In the first step, initial sensorimotor control indices are determined from an individual with a neurological impairment. After determining the sensorimotor control indices, the individual completes sensorimotor activities to rehabilitate the identified impairments. These sensorimotor treatment activities may be completed while the individual is in the HMD or in the physical, real world environment. The individual repeats the sensorimotor activities over time as exercises to rehabilitate, eliminate, reduce, or maintain the level of the neurological impairments identified. After completing one or more of the sensorimotor activities, the individual repeats the sensorimotor control tests and subsequent sensorimotor control indices for a specific sensorimotor control test are generated. These subsequent sensorimotor control indices are then compared to the initial sensorimotor control indices to determine the extent of recovery from the neurological impairment.

In contrast to traditional clinical practice, where it is very difficult to completely control a person's visual attention to test elements of sensorimotor control and the tests often lack objectivity because of the inability to measure subtle eye movements, reaction time, etc., the virtual immersive environment described herein beneficially captures the entirety of the user's visual field and thus their visual attention, and captures an individual's response data, thereby permitting objective testing. Moreover, as mentioned above, a remote healthcare provider 220 or examiner may use a remote computer device to monitor and control the virtual immersive environment and the sensorimotor tests presented to the individual. The remote healthcare provider may also make adjustments in real time and provide feedback and/or assessments of the user's test grade 218.

Although many of the examples described herein made in reference to testing of a neurological impairment associated with concussion injuries, the disclosure is not so limited and may include the testing of neurological impairment associated with any neurological event.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1—Detection of Neurological Impairments after a Transient/Acute Injury with the VIST Device

A concussion, synonymous with mild TBI, is a transient neurological injury for most people. It has been estimated that 80-90% of concussion injuries experience clinical resolution of signs and symptoms within an expected 10-30 day timeframe. At that time, most individuals resume normal activities, including sporting and military activities.

Unlike moderate and severe TBI, which produce frank structural deformities, a concussion is invisible to standard diagnostic imaging tools and is therefore, a very complicated diagnosis to make. Concussion is not detectably structural but rather completely functional in its presentation. Because of this, there is no gold standard in the diagnosis of concussion. Medical professionals must rely on their experience using patient reports and a constellation of signs and symptoms to derive a diagnosis. There are a variety of commonly used, non-instrumented clinical tools to help identify the presence of a concussion, including computerized neurocognitive tests, balance assessments, and attention-based oculomotor tests. Each of these examine one domain of brain function and require baseline assessment (i.e., a record of individual performance on the test prior to any injury) to compare to post-injury performance, which aids in the determination of what is abnormal for that individual. In addition to the impracticality of having baseline data from which to draw for each person in the population, concussions result in a variety of impairments, and every concussion presents differently. Therefore, completion of a test for just one brain function (e.g., cognition) does not adequately examine the range of additional brain functions that may be impaired.

Sensorimotor control impairments are very common after an ABI such as concussion. Although the medical community is aware of this, a thorough examination of sensorimotor control is not a component of standard evaluation and care. A primary reason is that sensorimotor examination is nuanced, can be subjective, and is often qualitative in its application. Additionally, several specialty or sub-specialty areas (i.e., neurology, ophthalmology, physical therapy, and others) contribute to the breadth of expertise required for a truly comprehensive examination. This has made it difficult for non-specialist healthcare providers to translate and implement into routine practice. A second reason is that there is inadequate access to these types of specialists in rural areas of the U.S., where examination is severely limited because the current compatible technology does not permit clinical examination of the oculomotor control and vestibular systems. Examination of these systems requires that a clinician has a clear view of the eyes during the completion of specific clinical tests.

In this example, 8 tests of sensorimotor control are completed with the VIST device and system in a non-concussed population (n>500) on an individual suspected of an acute concussion. Acutely concussed is defined as a person with an incident concussion no greater than 3 months from date of injury (n>50). All 8 tests are completed once by the participants. In this project, the data gathered is used: 1) to identify the presence of sensorimotor control impairment on each test independently and obtain a test grade, and 2) to calculate a global index and symptom indices across the data from one or more of the sensorimotor control tests to identify persons with a concussion from persons without a concussion.

For each test, 18-26 types of data are collected at 90 measurements per second. This is to include object and response data. Symptom assessment at the beginning of the test set and after each test enables the collection of symptom data. Data are collected through sensors on the IVR device, including one or more of, eye trackers, gyroscopes, accelerometers, magnetometers, (on the headset and in peripheral controllers), or activation by selection of the hand controller. Some of the collected data includes continuous eye-tracking to enable the calculation of measures including latency, movement accuracy, conjugate eye control, visual fixation stability, number of blinks, and pupil size. Because all tests are completed while standing, tri-planar postural sway data is also collected. Additional tests measure the number of correct selections, speed of selection, eye relative to head position, and other sensor-based measurements. Although the neuroanatomical processes in the brain of the individual are not being directly measured, the breadth of the neurologic functions challenged by the tests provides a more complete picture of overall neurologic health and degree of functional impairment compared to one singular stand-alone test.

Impairment is determined either by statistical analyses or by artificial intelligence function generated by one or more of: neural networks, gradient boosting, or support vector machines. The AI functions are generated on an existing participant population of at least 100 individuals not having a concussion, and the resulting computational function is used to assess new participants. These functions are updated on larger participant populations to generate more nuanced results.

An individual's data consists of rows, representing all recordings made at a single time step, and columns, which are the variables recorded. The columns are commonly called raw features, or just features. The variables and the number of rows are determined by the test.

For a given test, the data are subjected to a feature extraction/construction step, in which data is converted into a usable form by a function. These data are comprised of combination of one or more features. An example is the calculation of gaze position for an eye at some point, or the distance of the headset from a central point to represent postural sway, or calculation of the number of blinks during the test and their duration. In this way, it is seen that extracted features may form new features, with one time step for every time step in the collected data (long features), or single values that accumulate the behavior of the individual throughout the test (short features). The extracted features are collected into a single vector retaining the order of collection of each long feature. This long vector is the input to the AI function.

### Example 2—Detection of Neurological Impairments after a Chronic Injury with the VIST Device

Acquired brain injuries and neurological traumas of all types may produce sensorimotor control deficits. While some people recover, experiencing complete symptom resolution and absence of clinical signs indicative of neurological impairments, other people do not recover and experience chronic continuation of symptom provocation and clinical findings consistent with sensorimotor control deficits. People who continue to have symptoms and clinical findings indicative of neurological impairments 6 months after their initial injury are considered to be in the chronic phase of their condition. Individuals who experience continuation of functional neurological impairments are at risk for future injuries, especially if they resume athletic or military activities.

Although research has demonstrated increased risk for second concussion and extremity injury after one initial concussion, a thorough examination of sensorimotor control or consistent use of rehabilitative exercises to address sensorimotor control deficits are not part of standard care or return to activity guidelines. A primary reason is that sensorimotor examination is nuanced, can be subjective, and is often qualitative in its application. Additionally, several specialty or sub-specialty areas (i.e., neurology, ophthalmology, physical therapy, and others) contribute to the breadth of expertise required for a truly comprehensive examination. Even if a person is not attempting to return to sports or military environments after an ABI, identification of residual neurological impairments is important to initiate treatment, resolve lingering deficits, and ensure relative efficiency with everyday activities.

In this example, 8 tests of sensorimotor control are completed with the VIST device and system in a non-concussed population (n>500) and in a population with chronic ABI, defined as people with an ABI greater than 6 months from date of injury (n>50) who continue to have clinical signs or symptoms indicative of neurological impairment. All 8 tests are performed once by the participants. In this project, the data gathered are used to achieve two main outcomes: 1) an artificial intelligence to identify the presence of sensorimotor control impairment on each test independently and 2) a composite artificial intelligence across the data from one or more of the tests to identify persons with a chronic ABI from persons without an ABI.

The relevant features associated with individual performance from each of the tests that are used to identify neurological impairments in individuals with a chronic ABI. Secondarily, the relevant features across the sensorimotor control tests identify the probability of a chronic ABI.

### Example 3—Detection of Neurological Impairments in Individuals with a Progressive Neurological Disease with the VIST System

Progressive neurological diseases associated with aging are a general category of conditions characterized by the absence of a defining neurological trauma, but rather present as a slow, often insidious, onset of subtle neurological impairments that become more pronounced as time passes. Two of the most common types of central nervous system diseases in an aging population include Parkinson's disease and Alzheimer's disease. Early detection of these diseases is vitally important to permit directed treatment to prevent or slow progression and facilitate neurological health, where possible.

In general, a person must present with clinical signs or symptoms to initiate any type of diagnostic testing. Often, at the point diagnostic testing is initiated, disease processes have already taken a toll on the central nervous system, producing pronounced functional neurological impairments. This in-turn leads to secondary problems, including increased risk for falls, difficulty with executive cognitive activities, loss of independence with activities of daily living, and others. Because of this, new detection methods are needed to identify subtle changes in neurological function at the earliest stage of the disease process.

In this example, 8 tests of sensorimotor control are performed with the VIST device and system in a healthy population over age 60 (n>500) and in a population with early onset progressive neurological disease, defined as people over age 60 who have a medical diagnosis of a progressive central nervous system disease and who continue to function independently at home (n>50). All 8 tests are completed by the participants each 6 months for 6 years or until they are no longer able. The medical information collected include new onset diagnosis of a progressive neurological condition in the healthy subjects, so that the data is retrospectively analyzed to identify any early presentation of change in neurologic function.

In this project, the data gathered are used to achieve two main outcomes: 1) an artificial intelligence to identify changes in sensorimotor control test performance over time and 2) a composite artificial intelligence across the data from one or more of the tests to identify persons with a progressive neurological condition from persons without a progressive neurological condition.

### Example 4—Support

Epidemiology. Concussion is a form of traumatic brain injury (TBI) that occurs in response to mechanical insults due to falls, assaults, motor vehicle accidents, sports and recreation injuries, workplace injuries, and blast injuries from military actions.¹ The Centers for Disease Control and Prevention (CDC) estimates that 1.6 to 3.8 million sports and recreation brain injuries occur each year, 80-90% of which are considered mild TBIs, or concussion. Within the U.S. military, 80% of all service related TBIs are considered mild.^{2,3} In sports and military environments, concussion is one of the most difficult injury for physicians to diagnose and manage.^{3,4} This is largely because of the fast-paced environments in which concussions occur, the motivation to downplay injury, and the invisibility of the injury itself. But even off the field, concussion detection in a clinical setting continues to be a challenging endeavor; symptoms are non-specific, and no reliable biomarker has been found to date.⁴⁻⁷

Characteristics of Injury. Rapid acceleration-deceleration is the most common mechanism of injury for concussion. During these events, the anatomical orientation, joint mechanics, and relative weight of the human head to the cervical spine amplify rotational and shearing forces to the mid-axis of the brain. This includes micro-traumas to the long white matter tracts, which are vital for inter-hemispheric, sub-cortical, and cortical communication.^{8,9} Deficits in the functional connectivity of brain networks are frequently detected in individuals with a concussion using advanced neuroimaging and neurophysiological testing.⁵ However, deficits identified with advanced imaging are not uniform across people all with a concussion,¹⁰ and these types of tests are not routinely used in clinical practice for concussion detection.⁵

Clinical Assessments have Limited Diagnostic Utility. Clinical (non-instrumented) tests continue to be relied upon as standard practice to detect concussion. This generally includes a physical examination where the practitioner observes for gross evidence of impairment in one or more of the following: oculomotor and visual system; vestibular reflexes; standing postural control/balance; and coordination, with accompanying symptom report of the patient.^{3,4,6,11} Clinicians using these tests are only able to detect what they can perceive (i.e., gross abnormality) and what the patient is willing to admit (i.e., provocation of symptoms). Although subjective reports are helpful in detecting concussion, there remains a need for quantitative assessment tools that can better detect subtle functional impairments inherent to concussion.⁶ (symptoms aren't diagnostic) While some individuals with a concussion demonstrate clear clinical abnormalities, concussion is commonly far more nuanced and elusive to the naked eye. Furthermore, as with any neurological trauma, no two injuries present the same. It is this nuance, subtlety, and divergence of clinical presentation that accounts for why a gold standard has yet to be found.

Rurality Confers Additional Difficulties. Demand is high for alternative delivery solutions, both products and services, particularly in rural and economically stressed areas, where there is increased risk for concussion, limited access to care, and personal resources can be problematic.¹² In the rural poor areas of the US, there is inadequate access to specialists who can effectively diagnose and manage concussion. In places like Mississippi, for example, among 325 high schools, only 76 employ a full-time athletic trainer. Nationwide, no area is more impoverished than the Mississippi Delta, where not a single school has a full-time athletic trainer.¹³ Statewide, medical personnel presence is not mandated at games. In 2018, three high school football players died during game participation in Mississippi. In addition, Mississippi, has a shortage of healthcare providers equipped to address concussion, resulting in limited prevention efforts, missed on-field identification, and restricted evidence-based medical management.

These problems are not limited to Mississippi, but are present in many areas of the US. Use of a telehealth hub and spoke model, where a trauma center acts as the hub and the school's nurses office or local health clinic serves as the “spoke” facility, holds promise to increase access.^{14,15} Current use of telehealth for concussion management includes a video monitor for face to face conversation, discussion of symptoms, and gross assessments (to the degree possible) utilized for in-person management.¹⁶ At this time, telehealth as a solution for rural-poor health disparities in concussion care is severely limited in its diagnostic utility because current telehealth compatible technology does not exist to examine ocular and vestibular systems,¹⁶ which require a clinician to have a clear view of the eyes during the completion of specific clinical tests.

Sensorimotor Control=Sensory+Cognitive+Motor Processes. Sensorimotor control, including oculomotor and neuromotor control, is vital to the acquisition and execution of skilled human movement. When considered broadly but basically this includes inputs from the visual system (including perception and oculomotor control) to plan whole body movements in a feed-forward manner, the vestibular system to detect angular and linear acceleration of the head, providing feedback about movement and eliciting ocular and postural reflexes; and the somatic system to provide information across all joints and muscles in the body to permit proprioceptive and kinesthetic awareness. These inputs of sensory information are integrated and interpreted in the central nervous system and combined with cognitive processes, including attention and executive function, which results in directed efferent output to enable intended actions through precise motor control.¹⁷

Biomarkers of Sensorimotor Control Impairments after Concussion. Concussion frequently results in functional disturbances of the sensorimotor control system.¹⁸⁻²³ The connectivity and communication between the peripheral and cortical areas involved in sensorimotor control make it difficult to imagine that a concussion could occur without resultant disruption somewhere in the system. Within each human ability contributing to sensorimotor control defined below, research findings using sensor-based measurements are presented to detail the specific deficits and biomarkers which are indicative of concussion.

Visual System: Oculomotor control is mediated by neuronal connectivity of the midbrain, cerebellum, pons, and multiple areas of the cerebral cortex. Damage to one part or to the connections between areas can affect multiple components of vision functioning.⁹ Because of the speed of eye movements, most saccadic and pursuit deficits may be missed during traditional clinical examination tests Some of the most promising technologies to increase diagnostic accuracy for concussion based on deficits in eye movement control include video-based eye or pupil trackers.⁶ Findings presented below were detected with eye tracking technology, ranging between 60,²⁴ 90,⁹ 200,^{25,26} and 500 Hz.^{10,27} A recent systematic review found that within this range of sampling frequency, there was no relationship between significance of findings and sampling rate.²⁸

Smooth Pursuit: The complexity of the smooth pursuit eye movement system means that a number of smooth pursuit metrics have been found to distinguish concussed from non-concussed groups,^{27,29} but these distinguishing features vary across individuals with a concussion. Within this, however, there are patterns in performance variability that are markers of concussion.¹⁰ Synchronization between an individual's gaze and a moving target (most often a sinusoidal wave, which enables predictive smooth pursuit) has close ties to attention and adaptation in the brain.²⁷ Metrics of synchronization include phase lag time, average and peak velocity, positional errors, variability in eye position, and target prediction, which have been found in multiple studies to be impaired in individuals with concussion.^{9,25,27,29} Upper-limb visuomotor function has also been identified as a marker of concussion.^{25,30}

Saccades: Deficits in saccadic directional errors, spatial errors, fixation errors, prolonged reaction times in the anti-saccade task have been found with instrumented tests, which were not detectable on standard clinical assessment.^{24,26} Saccade amplitude and velocities during a sports-specific task have also been associated with concussion.²⁹ Additionally, differences in pupillary reflexes have been found during pro- and anti-saccade tests for people with neurological disorders,^{31,32} which have also been shown to be useful differentiators between concussed and non-concussed individuals.³³

Near-Point Convergence: Behaviors observed with near-point convergence (NPC) measurements after a concussion include fatigability (deterioration in performance between repeated tests), and convergence insufficiency (in upwards of 46% of people with concussion).³⁴ Deficits in NPC have been demonstrated to correlate with impaired reaction time,³⁴ with visual field deficits and vestibular dysfunction^{35,36} but not perfectly, showing evidence that divergent measures of sensorimotor control are necessary to identify specific deficits to diagnose concussion.

Peripheral Vision: Most eye-tracking research is focused on measures requiring central vision. Fewer studies have been conducted where use of peripheral vision was required. Research has shown that slowness of response to stimuli presented in the periphery of vision has been associated with micro-injuries to the white matter tracts in the corpus callosum, which is important in the transfer of information between brain hemispheres.⁸ Slowness in peripheral vision reaction time has been observed in multiple studies in people with a concussion compared to those who are non-concussed.^{8,37}

Cognition in Oculomotor Control and Visual Perception: In non-concussion populations, computer generated tasks utilizing visual stimuli to provoke goal directed eye movements are being increasingly used to screen and assess cognitive impairment, including executive dysfunction and attentional deficits in individuals with a variety of neurological diagnoses.³⁸⁻⁴⁰ Traditionally, these tests include oculomotor and visual perceptual tasks, such as smooth pursuit eye movements, pro- and anti-saccadic eye movements, fixation tasks, and visual search activities.³⁹ In individuals with neurological disorders, research findings have demonstrated a consistent link between oculomotor behaviors, neural mechanisms of brain function, and higher cognitive abilities.^{39,41-43} Research with individuals diagnosed with concussion has demonstrated that neural processing speed, including visually guided movements requiring the integration of cognitive and motor processes, are slowed.⁴⁴ Specific deficits in processing speed,⁴⁵ executive function,²⁷ visuospatial abilities,^{35,36} and visual attention^{10,27} have been demonstrated after concussion as well.

Vestibular System and Coordination: Concussion can not only result in micro-trauma to the central structures of the vestibular system but can also injure peripheral vestibular structures, particularly the otolith organs.⁴⁶ The instrumented Dynamic Visual Acuity test, a test of visual stability during head movement; has produced evidence of impairment in the stability of the vestibulo-ocular reflex (VOR) and retinal-slip, indicative of semi-circular canal and otolith organ dysfunction.⁴⁷ Additionally, research has demonstrated deficits in the use of the vestibular system for vertical perception and postural control as well as an inability to cognitively over-ride (cancel) the vestibular-ocular reflex (VOR) duffing head movements.⁴⁸

Proprioception and Postural Control: Balance and postural control are classically used to detect concussion. Sensor-based measures, however, have enhanced the objectivity of these tests and have revealed specific deficits. Multiple studies have demonstrated differences in balance during a dynamic visual motion tests based in virtual reality,^{49,50} even when clinical based assessments of balance were normal.⁵¹ Further, inertial sensor-based measures of standing balance are able to detect acute concussion when clinical tests of balance were not.⁵² Interestingly, center of pressure measurements on a force plate have also indicated specific differences in postural control in athletes with normal postural stability,⁵³ including decreased sway velocity during quiet standing.²³ Further, static balance, as measured by the instrumented Sensory Organization Test (SOT), demonstrates abnormal and increased postural sway for all conditions, particularly where the vestibular and somatosensory system are required.⁴⁸

### Example 5—Central and Peripheral Attention in Virtual Reality: Test of Visual Efficiency for Concussion Detection

Background: The human visual system is made up of two parts, central and peripheral. Relatively speaking, if the visual field is 170 degrees, 70 degrees is considered central, and 100 degrees is peripheral. These parts are quite different in their cellular composition, role in visual processing, and neural processes contributing to their function. Although the visual cortex processes central and peripheral information simultaneously, the information is processed independently, enabling the two parts of vision to be used and interpreted differently.

Peripheral vision allows us to see and respond to objects lateral to our body, gives us a sense of spatial perception in crowded areas, and enables us to view objects moving towards or around us. Humans remain consciously aware of what is being processed through their central vision, while they are subconsciously aware of what is being processed through their peripheral vision until there is a reason to direct conscious attention to it. In other words, the cortical processing of peripheral information causes a shift in central focus, when needed.

In complex, changing, dangerous, or fast-paced environments, both fields of vision are imperative to respond to changes in the environment, plan goal directed movement, avoid potential impacts, and ensure personal safety. Notably, peripheral vision is exceedingly important for participation in contact sports, while operating motorized vehicles or aircraft, and during military operations.

Measures of vision and oculomotor control have become key interests in research exploring biomarkers of mild traumatic brain injury/concussion. Most eye-tracking research is focused on measures requiring central vision. Fewer studies have been conducted where use of peripheral vision was required. The purpose of this analysis was to describe differences in performance between individuals with an acute concussion and controls on a novel test of peripheral vision delivered via a head mounted immersive virtual reality (IVR) device and to discuss the value of this test in the detection of concussion.

Methods: This study is a matched case-control, nested within a large cohort. Participants aged 18-40 with and without an acute concussion were enrolled from five universities in Mississippi. Determination of concussion was based on physician diagnosis. A propensity score matching was performed to identify non-concussed participants who had similar baseline covariates to concussed participants. First, the propensity score was estimated with a multivariate logistic regression model. Sex, race, number of hours slept the night before the test, and varsity athlete status were considered predictors.

In an effort to design a new medical device for decision support in the detection of concussion, eight sensorimotor control tests were developed to be administered in a 3-dimensional IVR environment. These tests produce a sensory stimulus to elicit a measurable voluntary or reflexive motor response, necessitate decision-making, visual attention and perception, and are completed while the individual is standing. This increases the complexity of the tests and brain processes required for success. During the completion of these tests, 18-26 distinct types of discrete data were continuously collected at 90 Hz using non-calibrated sensors, including an eye tracker.

The peripheral vision test includes one central object and six peripheral objects, three each on the left and right sides in a vertical column (up, center, and down). The test requires maintenance of focus on the center object and use of peripheral vision identify and select the matching side object with the hand-controller. After every selection, the shapes and colors of all the objects change and the side objects move further into the periphery. This is done for 60 repetitions (i.e., episodes).

We compared the test performance between matched pairs on the following outcomes: number of episodes where the eyes remained on the central object (i.e., a valid episode), correctness among valid episodes, mean latency without penalty for eye movement off the center (i.e., cheating), and mean latency with a penalty for eye movement off the center. The penalty was assigning the maximum allowable time (2000 milliseconds) to each episode where the individual moved their eyes off center. For each outcome, the overall performance was evaluated across episodes 2-60 as well as sub-sections of episodes 2-15, 16-30, 31-45, and 46-60. As we have multiple non-concussed participants for each concussed participant, comparisons were conducted between average test results of matched non-concussed participants and test results of concussed participants through paired t-test and Wilcoxon signed-rank test.

Results: In total, 611 participants have enrolled in the cohort. Within this, 24.4% are non-white, 49.7% are female, and 25.8% are varsity collegiate athletes. Within the matched sample, 13 participants with and concussion and 26 participants without a concussion were included in the analysis. Of these, 38.5% were Black, 36% were female, and 84.6% were varsity collegiate athletes. The concussed participants matched with two non-concussed participants by optimal propensity score matching with an absolute distance of less than 0.5, which shows balanced characteristics between matching pairs.

For the performance on the peripheral vision test, there was a significant difference in the number of valid episodes in the furthest periphery (episodes 46-60; mean 14.81 vs. 12.77; p=0.02). Among the valid episodes, there was no difference in correctness of peripheral selection between groups overall or for any of the sub-sections. There was a significantly longer latency overall when cheating episodes were penalized (mean 864.44 ms (SD 84.59) vs. 1039.83 ms (SD 219.67)) and were not penalized (mean 877.37 ms (SD 86.39) vs. 1107.33 ms (SD 250.75)) for the non-concussed and concussed groups, respectively. These significant differences in mean latency between groups were detected in the subsections of episodes 16-30, 31-45, and 46-60, and the differences in latency between groups became larger in the later episodes.

Conclusions: This single test of peripheral vision on a matched sample of concussed cases and non-concussed controls demonstrated several notable results. First, individuals with a concussion had fewer valid episodes in the last 25% of the test than controls. This indicates that at the end range of peripheral vision, their ability to efficiently process concurrent central and peripheral visual information was impaired, requiring the use of central vision to locate the correct object. Second, individuals with a concussion required upwards of 200 milliseconds more time to select the matching object in the periphery for the lateral 75% of the test than the controls. Third, individuals with a concussion did not make more selection errors than the non-concussed. Together with the prolonged latency, this indicates that after a concussion, peripheral vision perception is accurate, but the cortical processing time is delayed.

Peripheral vision reaction time (PVRT) has been described in two previous studies, which found increased PVRT among individuals with a concussion compared to those who were non-concussed. Our results demonstrate that IVR can be used to deliver a novel clinical peripheral vision test and produce corroborating findings. Our results, however, further elucidate specific deficits in peripheral vision processing after concussion. This is particularly important when designing rehabilitative programs to target specific deficits.

The IVR offers a portable solution, an optimally controlled environment, strong repeatability, sensor-based metrics of performance, and potential to be integrated within a telehealth model of care. These features are particularly important in military and rural environments, where specialist care is not typically available.

The relevant features associated with an individual's performance from each of the tests are used to identify neurological impairments in an individual with a progressive neurological disease. Secondarily, the relevant features across the sensorimotor control tests predict the probability, or likelihood that a person is going to be diagnosed with a progressive neurological disease.

### REFERENCES

All patents, patent applications, published applications and publications, GenBank sequences, databases, websites, and other published materials referred to throughout this specification are, unless noted otherwise, incorporated by reference to the same extent as if each was specifically and individually indicated to be incorporated by reference, including the references set forth in the following list:
1. Brain Injury Association of America. Traumatic Brain Injury Causes. Jul. 5, 2020. Accessed 07/05, 2020. https://www.biausa.org/brain-injury/about-brain-injury/basics/overview
2. Holtkamp M D, Grimes J, Ling G. Concussion in the Military: an Evidence-Base Review of mTBI in US Military Personnel Focused on Posttraumatic Headache. Current Pain and Headache Reports. 2016; 20(6)doi:10.1007/s11916-016-0572-x
3. McCrory P, Meeuwisse W, Dvorak J, et al. Consensus statement on concussion in sport-the 5th international conference on concussion in sport held in Berlin, October 2016. Br J Sports Med. Apr. 26, 2017; doi:10.1136/bj sports-2017-097699
4. Herring S, Kibler W B, Putukian M, et al. Selected issues in sport-related concussion (SRC|mild traumatic brain injury) for the team physician: a consensus statement. Br J Sports Med. November 2021; 55(22):1251-1261. doi:10.1136/bjsports-2021-104235
5. Wilkerson G B, Nabhan D C, Crane R T. Concussion History and Neuromechanical Responsiveness Asymmetry. Journal of athletic training. Jun. 23, 2020; 55(6):594-600. doi:10.4085/1062-6050-0401.19
6. Ferris L M, Kontos A P, Eagle S R, et al. Utility of VOMS, SCAT3, and ImPACT Baseline Evaluations for Acute Concussion Identification in Collegiate Athletes: Findings From the NCAA-DoD Concussion Assessment, Research and Education (CARE) Consortium. The American Journal of Sports Medicine. 2022:03635465211072261. doi:10.1177/03635465211072261
7. Pillai C, Gittinger J W, Jr. Vision Testing in the Evaluation of Concussion. Seminars in ophthalmology. 2017; 32(1):144-152. doi:10.1080/08820538.2016.1228412
8. Womack K B, Paliotta C, Strain J F, et al. Measurement of Peripheral Vision Reaction Time Identifies White Matter Disruption in Patients with Mild Traumatic Brain Injury. Journal of neurotrauma. Apr. 15, 2017; 34(8):1539-1545. doi:10.1089/neu.2016.4670
9. Hunfalvay M, Murray N P, Mani R, Carrick F R. Smooth Pursuit Eye Movements as a Biomarker for Mild Concussion within 7-Days of Injury. Brain Injury. 2021/12/06 2021; 35(14):1682-1689. doi:10.1080/02699052.2021.2012825
10. Maruta J, Suh M, Niogi S N, Mukherjee P, Ghajar J. Visual tracking synchronization as a metric for concussion screening. The Journal of head trauma rehabilitation. July-August 2010; 25(4):293-305. doi:10.1097/HTR.0b013e3181e67936
11. Mucha A, Collins M W, Elbin R J, et al. A Brief Vestibular/Ocular Motor Screening (VOMS) assessment to evaluate concussions: preliminary findings. Am J Sports Med. October 2014; 42(10):2479-86. doi:10.1177/0363546514543775
12. Yue J K, Upadhyayula P S, Avalos L N, Phelps R R L, Suen C G, Cage T A. Concussion and Mild-Traumatic Brain Injury in Rural Settings: Epidemiology and Specific Health Care Considerations. J Neurosci Rural Pract. January 2020; 11(1):23-33. doi:10.1055/s-0039-3402581
13. Pryor R R, Casa D J, Vandermark L W, et al. Athletic training services in public secondary schools: a benchmark study. Journal of athletic training. February 2015; 50(2):156-62. doi:10.4085/1062-6050-50.2.03
14. Vargas B B, Channer D D, Dodick D W, Demaerschalk B M. Teleconcussion: an innovative approach to screening, diagnosis, and management of mild traumatic brain injury. Article. Telemedicine journal and e-health: the official journal of the American Telemedicine Association. 2012; 18(10):803-806. doi:10.1089/tmj.2012.0118
15. Ochwat D. Teleconcussion care helps residents of rural Colorado. Hospitals & health networks. May 2017; 91(5):14-15.
16. Ellis M J, Boles S, Derksen V, et al. Evaluation of a pilot paediatric concussion telemedicine programme for northern communities in Manitoba. International journal of circumpolar health. December 2019; 78(1):1573163. doi:10.1080/22423982.2019.1573163
17. Peterka R J. Sensorimotor integration in human postural control. Journal of neurophysiology. September 2002; 88(3):1097-118.
18. Galea O A, Cottrell M A, Treleaven J M, O'Leary S P. Sensorimotor and Physiological Indicators of Impairment in Mild Traumatic Brain Injury: A Meta-Analysis. Neurorehabilitation and neural repair. February 2018; 32(2):115-128. doi:10.1177/1545968318760728
19. Samadani U, Li M, Qian M, et al. Sensitivity and specificity of an eye movement tracking-based biomarker for concussion. Concussion (London, England). March 2016; 1(1):Cnc3. doi:10.2217/cnc.15.3
20. Bin Zahid A, Hubbard M E, Lockyer J, et al. Eye Tracking as a Biomarker for Concussion in Children. Clin J Sport Med. September 2020; 30(5):433-443. doi:10.1097/j sm.0000000000000639
21. Samadani U, Ritlop R, Reyes M, et al. Eye movement tracking detects subclinical disconjugate gaze in structural and concussive brain injury. Conference Abstract. Journal of Neurosurgery. 2015; 122(6):A1544. doi:10.3171/2015.6.JNS.AANS2014abstracts
22. Doherty C, Zhao L, Ryan J, Komaba Y, Inomata A, Caulfield B. Quantification of postural control deficits in patients with recent concussion: An inertial-sensor based approach. Clinical biomechanics (Bristol, Avon). February 2017; 42:79-84. doi:10.1016/j.clinbiomech.2017.01.007
23. Hides J A, Franettovich Smith M M, Mendis M D, et al. A prospective investigation of changes in the sensorimotor system following sports concussion. An exploratory study. Musculoskeletal science & practice. June 2017; 29:7-19. doi:10.1016/j.msksp.2017.02.003
24. Heitger M H, Jones R D, Dalrymple-Alford J C, Frampton C M, Ardagh M W, Anderson T J. Motor deficits and recovery during the first year following mild closed head injury. Brain Injury. 2006/01/01 2006; 20(8):807-824. doi:10.1080/02699050600676354
25. Heitger M H, Anderson T J, Jones R D, Dalrymple-Alford J C, Frampton C M, Ardagh M W. Eye movement and visuomotor arm movement deficits following mild closed head injury. Brain: a journal of neurology. March 2004; 127(Pt 3):575-90. doi:10.1093/brain/awh066
26. Murray N G, Szekely B, Islas A, et al. Smooth Pursuit and Saccades after Sport-Related Concussion. Journal of neurotrauma. Jan. 15, 2020; 37(2):340-346. doi:10.1089/neu.2019.6595
27. Suh M, Kolster R, Sarkar R, McCandliss B, Ghajar J. Deficits in predictive smooth pursuit after mild traumatic brain injury. Neurosci Lett. Jun. 19, 2006; 401(1-2):108-13. doi:10.1016/j.neulet.2006.02.074
28. Wang C A, Brien D C, Munoz D P. Pupil size reveals preparatory processes in the generation of pro-saccades and anti-saccades. Eur J Neurosci. April 2015; 41(8):1102-10. doi:10.1111/ejn.12883
29. Bells S, Isabella S L, Brien D C, et al. Mapping neural dynamics underlying saccade preparation and execution and their relation to reaction time and direction errors. Human brain mapping. May 2020; 41(7):1934-1949. doi:10.1002/hbm.24922
30. Ayala N, Heath M. Executive Dysfunction after a Sport-Related Concussion Is Independent of Task-Based Symptom Burden. Journal of neurotrauma. Jul. 8 2020;doi:10.1089/neu.2019.6865
31. Pearce K L, Sufrinko A, Lau B C, Henry L, Collins M W, Kontos A P. Near Point of Convergence After a Sport-Related Concussion: Measurement Reliability and Relationship to Neurocognitive Impairment and Symptoms. Am J Sports Med. December 2015; 43(12):3055-61. doi:10.1177/0363546515606430
32. Alvarez T L, Kim E H, Vicci V R, Dhar S K, Biswal B B, Barrett A M. Concurrent vision dysfunctions in convergence insufficiency with traumatic brain injury. Optometry and vision science: official publication of the American Academy of Optometry. December 2012; 89(12):1740-51. doi:10.1097/OPX.0b013e3182772dce
33. Brahm K D, Wilgenburg H M, Kirby J, Ingalla S, Chang C Y, Goodrich G L. Visual impairment and dysfunction in combat-injured service members with traumatic brain injury. Optometry and vision science: official publication of the American Academy of Optometry. July 2009; 86(7):817-25. doi:10.1097/OPX.0b013e3181adff2d
34. Mallinson A, Maire R, Beyaert C, et al. Understanding and Managing Trauma-Induced Vestibular Deficits. J Int Adv Otol. November 2021; 17(6):559-565. doi:10.5152/iao.2021.21258
35. Zhou G, Brodsky J R. Objective vestibular testing of children with dizziness and balance complaints following sports-related concussions. Otolaryngology-head and neck surgery: official journal of American Academy of Otolaryngology-Head and Neck Surgery. June 2015; 152(6):1133-9. doi:10.1177/0194599815576720
36. Keller J, Krimly A, Bauer L, et al. A first approach to a neuropsychological screening tool using eye-tracking for bedside cognitive testing based on the Edinburgh Cognitive and Behavioural ALS Screen. Amyotroph Lateral Scler Frontotemporal Degener. August 2017; 18(5-6):443-450. doi:10.1080/21678421.2017.1313869
37. Tao L, Wang Q, Liu D, Wang J, Zhu Z, Feng L. Eye tracking metrics to screen and assess cognitive impairment in patients with neurological disorders. Neurol Sci. July 2020; 41(7):1697-1704. doi:10.1007/s10072-020-04310-y
38. Asato M R, Nawarawong N, Hermann B, Crumrine P, Luna B. Deficits in oculomotor performance in pediatric epilepsy. Epilepsia. February 2011; 52(2):377-85. doi:10.1111/j.1528-1167.2010.02787.x
39. Wolf K, Galeano Weber E, van den Bosch J J F, et al. Neurocognitive Development of the Resolution of Selective Visuo-Spatial Attention: Functional MRI Evidence From Object Tracking. Front Psychol. 2018; 9:1106. doi:10.3389/fpsyg.2018.01106
40. İşbilir E, çakir MP, Acartürk C, Tekerek A. Towards a Multimodal Model of Cognitive Workload Through Synchronous Optical Brain Imaging and Eye Tracking Measures. Front Hum Neurosci. 2019; 13:375. doi:10.3389/fnhum.2019.00375
41. Amador S C, Hood A J, Schiess M C, Izor R, Sereno A B. Dissociating cognitive deficits involved in voluntary eye movement dysfunctions in Parkinson's disease patients. Neuropsychologia. 2006; 44(8):1475-82. doi:10.1016/j.neuropsychologia.2005.11.015
42. Fueger C, Huddleston W E. Effects of concussions on visually guided motor actions: A literature review. Journal of clinical and experimental neuropsychology. December 2018; 40(10):1074-1080. doi:10.1080/13803395.2018.1458823
43. Colvin A C, Mullen J, Lovell M R, West R V, Collins M W, Groh M. The role of concussion history and gender in recovery from soccer-related concussion. Am J Sports Med. September 2009; 37(9):1699-704. doi:10.1177/0363546509332497
44. Wright W G, Tierney R T, McDevitt J. Visual-vestibular processing deficits in mild traumatic brain injury. Journal of vestibular research: equilibrium & orientation. 2017; 27(1):27-37. doi:10.3233/ves-170607
45. Slobounov S, Newell K, Sebastianelli W. Virtual reality graphics in a clinical assessment of concussion. Conference Abstract. Journal of Head Trauma Rehabilitation. 2008; 23(5):340. doi:10.1097/01.HTR.0000336846.06704.67
46. Slobounov S, Slobounov E, Newell K. Application of virtual reality graphics in assessment of concussion. Article. Cyberpsychology and Behavior. 2006; 9(2):188-191. doi:10.1089/cpb.2006.9.188
47. King L A, Mancini M, Fino P C, et al. Sensor-Based Balance Measures Outperform Modified Balance Error Scoring System in Identifying Acute Concussion. Annals of biomedical engineering. September 2017; 45(9):2135-2145. doi:10.1007/s10439-017-1856-y
48. Cavanaugh J T, Guskiewicz K M, Giuliani C, Marshall S, Mercer V, Stergiou N. Detecting altered postural control after cerebral concussion in athletes with normal postural stability. Br J Sports Med. November 2005; 39(11):805-11. doi:10.1136/bjsm.2004.015909

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method to detect neurological impairment of a user using examination of sensorimotor control, the method comprising:
positioning a head-mounted display on the user's head, the head-mounted display placing the user in a virtual or augmented reality environment;
presenting the subject with a software-generated object in the virtual or augmented reality environment;
providing instructions directing the user to execute one or more sensorimotor activities relating to the software-generated object within the virtual or augmented reality environment;
recording data during execution of the one or more objectives; and
determining if there is a neurological impairment based upon the recorded data;
wherein the method is at least partially implemented by a computer system comprising:
the head-mounted display;
one or more sensors for recording the data;
one or more processors; and
one or more hardware storage devices.

2. The method of claim 1, wherein the sensorimotor activities are selected from the group consisting of:
smooth pursuit;
convergence eye movement;
saccadic eye movement;
peripheral visual acuity;
object discrimination;
gaze stability;
head-eye coordination;
cervical neuromotor control; and
combinations thereof.

3. The method of claim 1, wherein the instructions are provided to the user as audio or visual instructions.

4. The method of claim 1, wherein the data includes one or more of object data, response data, or symptom data.

5. The method of claim 1, wherein the placing of the user in a virtual or augmented reality environment includes displaying a three-dimensional environment to the user through the head-mounted display.

6. The method of claim 1, wherein the head-mounted display includes a screen for each eye.

7. The method of claim 1, wherein the step of determining if there is a neurological impairment based upon the recorded data comprises:
generating a sensorimotor index from the recorded data;
assigning a test grade based upon the sensorimotor index; and
determining if the user has a neurological impairment based upon the test grade.

8. The method of claim 7, wherein the sensorimotor index is generated using statistical computation or machine learning artificial intelligence computation.

9. The method of claim 1, wherein the step of determining if there is a neurological impairment based upon the recorded data comprises:
generating a user sensorimotor index from the recorded data;
comparing the user sensorimotor index to at least one other sensorimotor index; and
determining if the user has a neurological impairment based upon the comparison with the at least one other sensorimotor index.

10. The method of claim 9, wherein the at least one other sensorimotor index is selected from the group consisting of a previous sensorimotor index generated from the user's prior data, a designated population without a known neurological impairment, and a designated population with a known neurological impairment.

11. The method of claim 9, wherein the user sensorimotor index and at least one other sensorimotor index are generated using statistical computation or machine learning artificial intelligence computation.

12. The method of claim 11, wherein the machine learning artificial intelligence makes a determination about the type of impairment for each sensorimotor activity or a combination of one or more of the sensorimotor activities when completed together;

13. The method of claim 1, wherein the one or more sensors comprise a hand tracking sensor for tracking position and movement of the user's hand within the virtual immersive environment relative to other objects displayed within the virtual immersive environment.

14. The method of claim 1, wherein the one or more sensors comprise an accelerometer head sensor, a gyroscope head sensor, an eye tracking device, a magnetometer, or combination thereof.

15. The method of claim 1, wherein the user is neurologically impaired due to trauma, vascular, aging, or other physiological processes.

16. A device configured for testing sensorimotor control to detect neurological impairment of a user, the device comprising:
a head-mounted display comprising:
a screen for each eye, the screens being configured to display a virtual immersive environment to the user;
an audio system configured to provide instructions to the user for executing one or more sensorimotor activities within the virtual immersive environment;
an eye tracking system; and
at least one sensor configured to record data during the sensorimotor activities;
one or more processors; and
one or more hardware storage devices having stored thereon computer-executable instructions which are executable by the one or more processors to cause the display of the virtual immersive environment and recording of the data.

17. The device of claim 16, wherein the at least one sensor comprise an accelerometer head sensor, a gyroscope head sensor, an eye tracking device, a magnetometer, or combination thereof.

18. The device of claim 16, wherein the one or more sensorimotor activities comprise one or more of smooth pursuit, saccade, near point convergence, peripheral vision acuity, object discrimination, gaze stability, head-eye coordination, or cervical neuromotor control.

19. The device of claim 16, wherein the eye tracking system is selected from the group consisting of a single tracker for both eyes and two trackers for each eye.

20. A hardware storage device having stored thereon computer-executable instructions which are executable by one or more processors of a computer system to cause the computer system to:
record a sensorimotor performance metric of a user;
display a virtual immersive environment to the user by way of the head-mounted display with one screen for an eye;
provide instructions directing the user to execute one or more sensorimotor activities within the virtual immersive environment, and recording object, response, or symptom data of the user during execution of the one or more sensorimotor activities;
based on the recorded object, response, or symptom data, determine sensorimotor performance metric of the user indicating the user's proficiency in executing the one or more sensorimotor activities;
use statistical testing to determine if there is an impairment of sensorimotor control.
